(19) United States
(12) Patent Application Publication
(10) Pub. No.: US 2021/0030718 A1
(43) Pub. Date: Feb. 4, 2021

(54) COMBINATIONS FOR TREATING CANCER

(71) Applicant: ASTRAZENECA AB, Södertälje (SE)

(72) Inventors: Scott BOIKO, Waltham, MA (US); Justin CIDADO, Waltham, MA (US); Lisa DREW, Waltham, MA (US); Theresa PROIA, Waltham, MA (US); Maryann SAN MARTIN, Waltham, MA (US)

(21) Appl. No.: 17/047,931

(22) PCT Filed: Apr. 29, 2019
(86) PCT No.: PCT/IB2019/053491

§ 371 Date: Oct. 15, 2020

Related U.S. Application Data
(60) Provisional application No. 62/664,356, filed on Apr. 30, 2018.

(51) Int. Cl. A61K 31/4155; A61K 31/4985; A61P 35/00

(52) U.S. Cl. CPC A61K 31/4155; A61P 35/00; A61K 31/4985

(57) ABSTRACT

Disclosed are methods of treating cancer comprising administering 17-chloro-5,13,14,22-tetramethyl-28-oxa-2,9-dithia-5,6, 12,13,22-pentaazaheptacyclo[27.7.1.1^{4,7}.0^{11,15}.0^{16,21}.0^{20,24}.0^{30,35}]octatriaconta-1 (37), 4(38), 6, 11, 14, 16, 18,20,23,29,31,33,35-tridecaene-23-carboxylic acid, a pharmaceutically acceptable salt thereof; and acalabrutinib, or a pharmaceutically acceptable salt thereof.

### BACKGROUND

Bruton tyrosine kinase (BTK), a member of the TEC family of kinases, is an important component in the B-cell receptor (BCR) signaling pathway, where it sits between the BCR and downstream survival signals. BTK is expressed in cells of hematopoietic lineage, except for T cells, and is upregulated in chronic lymphocytic leukemia (CLL) cells relative to normal B cells. BTK is also essential for proliferation and survival of some oB-cell malignancies. In particular, knockdown of BTK induces tumor cell death in primary CLL cells and lymphoma cell lines that are dependent on BCR signaling. Furthermore, genetic ablation of BTK inhibits disease progression in mouse models of CLL, indicating its continued importance for B-cell malignancies.

Myeloid Cell Leukemia 1 (Mcl-1) is an important anti-apoptotic member of the BCL-2 family of proteins and a master regulator of cell survival. Amplification of the Mcl1 gene and/or overexpression of the Mcl-1 protein has been observed in multiple cancer types and is commonly implicated in tumor development. In fact, MCL1 is one of the most frequently amplified genes in human cancer. In many malignancies, Mcl-1 is a critical survival factor and it has been shown to mediate drug resistance to a variety of anti-cancer agents.

Mcl-1 promotes cell survival by binding to pro-apoptotic proteins like Bim, Noxa, Bak, and Bax and neutralizing their death-inducing activities. Inhibition of Mcl-1 thereby releases these pro-apoptotic proteins, often leading to the induction of apoptosis in tumor cells dependent on Mcl-1 for survival. Therapeutically targeting Mcl-1 alone or in combination with other therapies, therefore, is a promising strategy to treat a multitude of malignancies and to overcome drug resistance in many human cancers.

### SUMMARY

In some embodiments, disclosed is a method of treating cancer comprising administering to a subject in need thereof an effective amount of 17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (Compound A) or a pharmaceutically acceptable salt thereof; and an effective amount of acalabrutinib, or a pharmaceutically acceptable salt thereof.

In some embodiments, disclosed is a method of treating cancer comprising administering to a subject in need thereof an effective amount of (Rₐ)-17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (AZD5991), or a pharmaceutically acceptable salt thereof, and acalabrutinib, or a pharmaceutically acceptable salt thereof.

In some embodiments, disclosed is 17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (Compound A) or a pharmaceutically acceptable salt thereof for use in the treatment of cancer in a subject, wherein said treatment comprises the separate, sequential or simultaneous administration of Compound A, or a pharmaceutically acceptable salt thereof, and acalabrutinib, or a pharmaceutically acceptable salt thereof, to said subject.

In some embodiments, disclosed is (Rₐ)-17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (AZD5991), or a pharmaceutically acceptable salt thereof, for use in the treatment of cancer in a subject, wherein said treatment comprises the separate, sequential or simultaneous administration of AZD5991, or a pharmaceutically acceptable salt thereof, and acalabrutinib, or a pharmaceutically acceptable salt thereof, to said subject.

In some embodiments, disclosed is acalabrutinib, or a pharmaceutically acceptable salt thereof, for use in the treatment of cancer in a subject, wherein said treatment comprises the separate, sequential or simultaneous administration of acalabrutinib, or a pharmaceutically acceptable salt thereof, and 17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (Compound A), or a pharmaceutically acceptable salt thereof.

In some embodiments, disclosed is acalabrutinib, or a pharmaceutically acceptable salt thereof, for use in the treatment of cancer in a subject, wherein said treatment comprises the separate, sequential or simultaneous administration of acalabrutinib, or a pharmaceutically acceptable salt thereof, and (Rₐ)-17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (AZD5991), or a pharmaceutically acceptable salt thereof.

In some embodiments, disclosed is a kit comprising: a first pharmaceutical composition comprising acalabrutinib, or a pharmaceutically acceptable salt thereof, and a pharmaceutically acceptable carrier; and a second pharmaceutical composition comprising Compound A, or a pharmaceutically acceptable salt thereof, and a pharmaceutically acceptable carrier.

In some embodiments, disclosed is a kit comprising: a first pharmaceutical composition comprising acalabrutinib, or a pharmaceutically acceptable salt thereof, and a pharmaceutically acceptable carrier; and a second pharmaceutical composition comprising AZD5991, or a pharmaceutically acceptable salt thereof, and a pharmaceutically acceptable carrier.

### BRIEF DESCRIPTION OF THE DRAWINGS

FIG. 1 illustrates that acalabrutinib increases protein levels of pro-apoptotic proteins, including Bim and Bmf, in BTK inhibitor-sensitive DLBCL cell lines that leads to priming of cells for apoptosis.

FIG. 2 shows that the combination of AZD5991 with 24-hour acalabrutinib pre-treatment results in enhanced and rapid caspase activation in BTK inhibitor-sensitive cell lines (OCILy10 and TMD8).

FIG. 3 shows that combination of AZD5991 with acalabrutinib produces synergistic anti-tumor efficacy in the in vivo ABC-DLBCL model OCILy10.

### DETAILED DESCRIPTION

In some embodiments, disclosed is a method of treating cancer comprising administering to a subject in need thereof an effective amount of 17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (Compound A), or a pharmaceutically acceptable salt thereof; and an effective amount of acalabrutinib, or a pharmaceutically acceptable salt thereof.

The language “treat,” “treating” and “treatment” includes the reduction or inhibition of enzyme or protein activity related to Mcl-1, BTK or cancer in a subject, amelioration of one or more symptoms of cancer in a subject, or the slowing or delaying of progression of cancer in a subject. The language “treat,” “treating” and “treatment” also includes the reduction or inhibition of the growth of a tumor or proliferation of cancerous cells in a subject.

The language “inhibit,” “inhibition” or “inhibiting” includes a decrease in the baseline activity of a biological activity or process.

The term “cancer” includes, but is not limited to hematological malignancies such as acute myeloid leukemia (AML), multiple myeloma, mantle cell lymphoma (MCL), chronic lymphocytic leukemia (CLL), diffuse large B cell lymphoma (DLBCL), Burkitt's lymphoma and follicular lymphoma. In some embodiments, the cancer is a BTK-sensitive cancer. The term “BTK-sensitive cancer” refers to cancers that are susceptible to treatment with BTK inhibitors (e.g., acalabrutinib). In some embodiments, the cancer is DLBCL. In some embodiments, the cancer is activated B cell-like diffuse large B cell lymphoma (ABC-DLBCL).

The term “subject” includes warm-blooded mammals, for example, primates, dogs, cats, rabbits, rats, and mice. In some embodiments, the subject is a primate, for example, a human. In some embodiments, the subject is suffering from cancer. In some embodiments, the subject is in need of treatment (e.g., the subject would benefit biologically or medically from treatment).

The language “effective amount” includes an amount of acalabrutinib, or a pharmaceutically acceptable salt thereof, and Compound A, AZD5991 or pharmaceutically acceptable salts of Compound A and AZD5991, that will elicit a biological or medical response in a subject, for example, the reduction or inhibition of enzyme or protein activity related to Mcl-1, BTK or cancer; amelioration of symptoms of cancer; or the slowing or delaying of progression of cancer. In some embodiments, the language “effective amount” includes the amount of acalabrutinib, or a pharmaceutically acceptable salt thereof, and Compound A, AZD5991 or pharmaceutically acceptable salts of Compound A and AZD5991, that when administered to a subject, is effective to at least partially alleviate, inhibit, and/or ameliorate cancer or inhibit Mcl-1 or BTK, and/or reduce or inhibit the growth of a tumor or proliferation of cancerous cells in a subject.

Acalabrutinib, also referred to as ACP-196 or (S)-4-(8-amino-3-(1-but-2-ynoylpyrrolidin-2-yl)imidazo[1,5-a]pyrazin-1-yl)-N-(pyridin-2-yl)benzamide, has the formula:

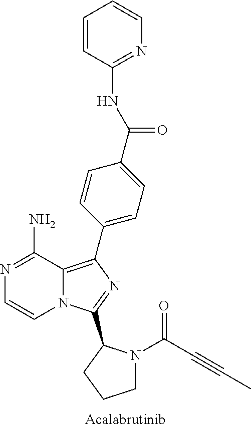 [SMILES: CC#CC(=O)N1CCC[C@H]1c1nc(-c2ccc(C(=O)Nc3ccccn3)cc2)c2c(N)nccn12]

Methods of making acalabrutinib are described in, for example, WO 2013/010868 which is incorporated by reference in its entirety. In some embodiments, acalabrutinib can be replaced by a different BTK inhibitor, or a pharmaceutically acceptable salt thereof, for example, ibrutinib (IMBRUVICA), spebrutinib (CC-292), zanabrutinib (BGB-3111) or tirabrutinib (ONO/GS-4059)

Compound A (17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid), solid forms of Compound A and methods of making Compound A are disclosed in International Patent Application Publication No. WO2017/182625, incorporated herein by reference in its entirety, and has the structure:

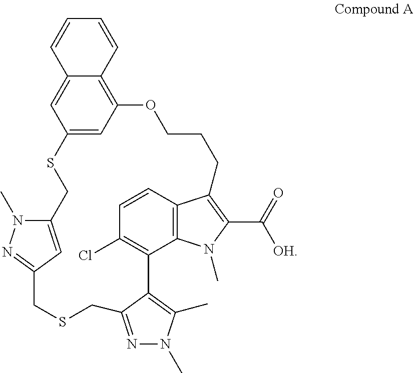 [SMILES: Cc1c2c(nn1C)CSCc1cc(n(C)n1)CSc1cc(c3ccccc3c1)OCCCc1c(C(=O)O)n(C)c3c-2c(Cl)ccc13]

In some embodiments, disclosed is a method of treating cancer comprising administering to a subject in need thereof an effective amount Compound A; and an effective amount of acalabrutinib. In some embodiments, disclosed is a method of treating cancer comprising administering to a subject in need thereof an effective amount of a pharmaceutically acceptable salt of Compound A; and an effective amount of acalabrutinib.

In some embodiments, disclosed is a method of treating cancer comprising administering to a subject in need thereof an effective amount of (Rₐ)-17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (AZD5991) or a pharmaceutically acceptable salt thereof; and an effective amount of acalabrutinib. The structure of AZD5991 is:

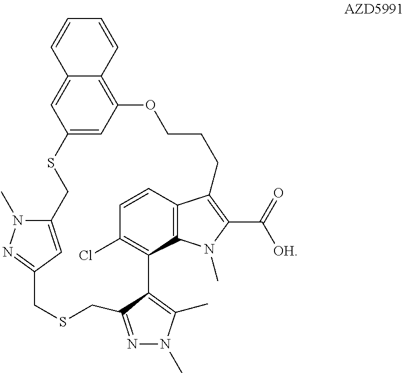

AZD5991, methods of making AZD5991 and solid forms of AZD5991 are disclosed in International Patent Application Publication No. WO2017/182625, which is incorporated herein by reference in its entirety.

In some embodiments, disclosed is a method of treating cancer comprising administering to a subject in need thereof an effective amount of AZD5991 and an effective amount of acalabrutinib. In some embodiments, disclosed is a method of treating cancer comprising administering to a subject in need thereof an effective amount of AZD5991 and an effective amount of acalabrutinib, or a pharmaceutically acceptable salt thereof. In some embodiments, disclosed is a method of treating cancer comprising administering to a subject in need thereof an effective amount of AZD5991, or a pharmaceutically acceptable salt thereof, and an effective amount of acalabrutinib. In some embodiments, disclosed is a method of treating cancer comprising administering to a subject in need thereof an effective amount of AZD5991, or a pharmaceutically acceptable salt thereof, and an effective amount of acalabrutinib, or a pharmaceutically acceptable salt thereof.

The language “pharmaceutically acceptable salt” includes acid addition or base salts that retain the biological effectiveness and properties of Compound A, AZD5991 or acalabrutinib, which typically are not biologically or otherwise undesirable. In many cases, Compound A or AZD5991 are capable of forming acid and/or base salts by virtue of the presence of basic and/or carboxyl groups or groups similar thereto. In one embodiment, the pharmaceutically acceptable salt includes quaternary ammonium salts.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethanedisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulfate, naphthoate, napsylate, nicotinate, nitrate, octadecanoate, oleate, oxalate, palmitate, palmoate, phosphate/hydrogen phosphate/dihydrogen phosphate, polygalacturonate, propionate, stearate, succinate, subsalicylate, sulfate/hydrogensulfate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoroacetic acid, sulfosalicylic acid, and the like.

Pharmaceutically acceptable base addition salts can be formed with inorganic and organic bases. Inorganic bases from which salts can be derived include, for example, ammonia and salts of ammonium and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine. In some aspects, the pharmaceutically acceptable salt of AZD5991 is the sodium salt. In some aspects, the pharmaceutically acceptable salt of AZD5991 is the meglumine salt.

In some embodiments, disclosed is a method of treating cancer comprising administering to a subject in need thereof an effective amount a solid form of AZD5991, or a pharmaceutically acceptable salt thereof; and an effective amount of acalabrutinib. In some embodiments, disclosed is a method of treating cancer comprising administering to a subject in need thereof an effective amount of a solid form of AZD5991; and an effective amount of acalabrutinib. In some embodiments, disclosed is a method of treating cancer comprising administering to a subject in need thereof an effective amount of a pharmaceutically acceptable salt of a solid form of AZD5991; and an effective amount of acalabrutinib.

The term “solid form” includes polymorphs, crystalline salts, solvates, hydrates and amorphous forms of AZD5991. The term “polymorph” includes crystalline materials that have the same chemical composition but different molecular packing. The language “crystalline salt” includes crystalline structures with the same chemical materials, but incorporating acid or base addition salts within the molecular packing of the crystalline structure. The term “solvate” includes crystalline structures of the same chemical material, but incorporating molecules of solvent within the molecular packing of the crystalline structure. The term “hydrates” includes crystalline structures of the same chemical material, but incorporating molecules of water within the molecular packing of the crystalline structure. The language “amorphous form” includes compounds of the same molecular material but without the molecular order of a crystalline structure (e.g., polymorph, crystalline salt, solvate or hydrate) of the same molecular material.

In some embodiments, the solid form of AZD5991 is Form A, Form B, Form C, Form D, Form E, Form F, the sodium salt of AZD5991 or the meglumine salt of AZD5991, as disclosed in International Patent Application Publication No. WO2017/182625, incorporated herein by reference in its entirety.

In some embodiments, disclosed is Compound A or a pharmaceutically acceptable salt thereof for use in the treatment of cancer in a subject, wherein said treatment comprises the separate, sequential or simultaneous administration of Compound A, or a pharmaceutically acceptable salt thereof, and acalabrutinib to said subject.

In some embodiments, disclosed Compound A for use in the treatment of cancer in a subject, wherein said treatment comprises the separate, sequential or simultaneous administration of Compound A and acalabrutinib to said subject.

In some embodiments, disclosed is a pharmaceutically acceptable salt of Compound A for use in the treatment of cancer in a subject, wherein said treatment comprises the separate, sequential or simultaneous administration of a pharmaceutically acceptable salt of Compound A and acalabrutinib to said subject.

In some embodiments, disclosed is AZD5991, or a pharmaceutically acceptable salt thereof, for use in the treatment of cancer in a subject, wherein said treatment comprises the separate, sequential or simultaneous administration of AZD5991, or a pharmaceutically acceptable salt thereof; and acalabrutinib to said subject.

In some embodiments, disclosed is AZD5991 for use in the treatment of cancer in a subject, wherein said treatment comprises the separate, sequential or simultaneous administration of AZD5991 and acalabrutinib to said subject.

In some embodiments, disclosed is a pharmaceutically acceptable salt of AZD5991 for use in the treatment of cancer in a subject, wherein said treatment comprises the separate, sequential or simultaneous administration of a pharmaceutically acceptable salt of AZD5991 and acalabrutinib to said subject.

In some embodiments, disclosed is a solid form of AZD5991, or a pharmaceutically acceptable salt thereof, for use in the treatment of cancer in a subject, wherein said treatment comprises the separate, sequential or simultaneous administration of a solid form of AZD5991, or a pharmaceutically acceptable salt thereof, and acalabrutinib to said subject.

In some embodiments, disclosed is a solid form of AZD5991 for use in the treatment of cancer in a subject, wherein said treatment comprises the separate, sequential or simultaneous administration of a solid form of AZD5991, and acalabrutinib to said subject.

In some embodiments, disclosed is a pharmaceutically acceptable salt of a solid form of AZD5991 for use in the treatment of cancer in a subject, wherein said treatment comprises the separate, sequential or simultaneous administration of a pharmaceutically acceptable salt of a solid form of AZD5991, and acalabrutinib to said subject.

In some embodiments, disclosed is a kit comprising a first pharmaceutical composition comprising acalabrutinib and a pharmaceutically acceptable carrier; and a second pharmaceutical composition comprising Compound A, or a pharmaceutically acceptable salt thereof, and a pharmaceutically acceptable carrier. In some embodiments, the pharmaceutical composition comprising acalabrutinib is a capsule further comprising silicified microcrystalline cellulose, partially pregelatinized starch, magnesium stearate, and sodium starch glycolate. In some embodiments, the pharmaceutical composition comprising Compound A is a pharmaceutical composition disclosed in International Patent Application Publication No. WO2017/182625, incorporated herein by reference in its entirety.

In some embodiments, acalabrutinib is administered as a capsule. In some embodiments, acalabrutinib is administered in a 100 mg dose. In some embodiments, acalabrutinib is administered twice daily, for example, every 12 hours. In some embodiments, acalabrutinib is administered sequentially, simultaneously or simultaneously with Compound A, or a pharmaceutically acceptable salt thereof. In some embodiments, acalabrutinib is administered separately, sequentially or simultaneously with AZD5991 or a solid form of AZD5991, or pharmaceutically acceptable salts thereof. In some embodiments, acalabrutinib is administered about 24 hours prior to administration of Compound A, AZD5991, or solid forms of AZD5991, or pharmaceutically acceptable salts thereof. In some embodiments, acalabrutinib is administered in an amount effective to inhibit BKT. In some embodiments, acalabrutinib is administered as a single dose. In some embodiments, acalabrutinib is administered as multiple doses.

In some embodiments, Compound A, AZD5991 or a solid form of AZD5991, or pharmaceutically acceptable salts thereof, is administered intravenously. In some embodiments, Compound A, AZD5991 or a solid form of AZD5991, or pharmaceutically acceptable salts thereof, is administered about once per week. In some embodiments, Compound A, AZD5991 or a solid form of AZD5991, or pharmaceutically acceptable salts thereof, is administered in an amount effective to inhibit Mcl-1. In some embodiments, Compound A, AZD5991 or a solid form of AZD5991, or pharmaceutically acceptable salts thereof, is administered as a single dose. Compound A, AZD5991 or a solid form of AZD5991, or pharmaceutically acceptable salts thereof, is administered as multiple doses. In some embodiments, Compound A, AZD5991 or a solid form of AZD5991, or pharmaceutically acceptable salts thereof, is administered separately, sequentially or simultaneously with acalabrutinib.

### EXAMPLES

### Example 1: Acalabrutinib Primes BTK Inhibitor-Sensitive Cancer Cells Lines to Die Through Increased Protein Levels of Pro-Apoptotic BH3-Only Proteins

Two ABC-DLBCL (OCILy10 and TMD8) cell lines and one GCB-DLBCL (Karpas422) cell line were treated with either vehicle (DMSO) or a 10-point, ½ log serially dilution of acalabrutinib for 72 hours with a CellTiter-Glo viability readout. The concentration at which 50% growth is inhibited (GI₅₀) was calculated using GraphPad Prism or GeneData, as shown in Table 1.

TABLE 1

| Cell Line | DLBCL Subtype | 72 hr GI₅₀ (μM) |
|---|---|---|
| OCILy10 | ABC | 0.01 |
| TMD8 | ABC | 0.06 |
| Karpas422 | GCB | >31 |

OCILy10, TMD8, and Karpas422 were treated with either vehicle or 100 nM acalabrutinib for 2-72 hours. Protein lysates were harvested at multiple time points in that interval, protein concentration was determined using the BCA Protein Assay Kit, and western blots were performed according to standard protocol to assess the effects on Bcl2 family protein levels. Each sample was normalized to vinculin as a loading control, and then protein levels of acalabrutinib-treated samples were calculated relative to those of vehicle-treated samples.

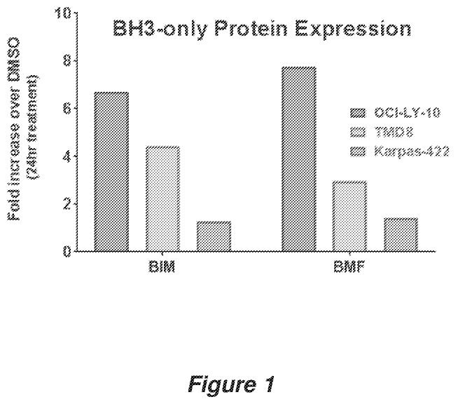

Each of the three cell lines were then treated with 100 nM acalabrutinib for 24 hours, and BH3-only protein levels were assessed. The levels of pro-apoptotic BH3-only proteins Bim and Bmf were increased in the two acalabrutinib-sensitive DLBCL cell lines relative to the insensitive cell line, priming the cells for apoptosis (FIG. 1). Acalabrutinib increases protein levels of pro-apoptotic proteins, including Bim and Bmf, in BTK inhibitor-sensitive DLBCL cell lines that leads to priming of cells for apoptosis. The results of Example 1 illustrate that diffuse large B-cell leukemia (DLBCL) cell lines display a differential sensitivity to acalabrutinib. A dose-response of acalabrutinib in three DLBCL cell lines shows two ABC-DLBCL cell lines, OCILy10 and TMD8, are sensitive to BTK inhibition while the GCB-DLBCL cell line, Karpas422, is not.

### Example 2: Combination of Acalabrutinib and AZD5991 Leads to Enhanced and Rapid Induction of Cell Death

Two BTK inhibitor-sensitive ABC-DLBCL cell lines (OCILy10 and TMD8) were treated for an 8-hour time course with either 1 μM AZD5991 alone or following a 24-hour pre-treatment with 100 nM acalabrutinib (since exposure for 24 hours was sufficient to induce maximal BH3-only protein levels). Cells were harvested for protein lysates at varying time points (0, 0.5, 1, 2, 4, and 8 hours) post-AZD5991 treatment, normalized for protein concentration using the BCA Protein Assay Kit, and run for western blots according to standard protocols. To ensure expected target engagement of acalabrutinib, the blots were probed for the proximal biomarker for activated BTK (pBTK Y223). To gauge the time to induction of apoptosis, cleaved caspase-3 was also assessed. A loading control (vinculin) was also utilized for normalization.

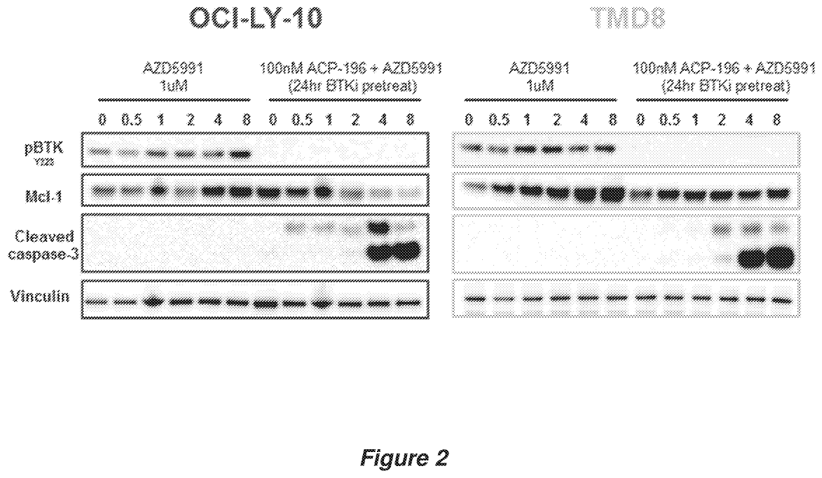

Since acalabrutinib increases protein levels of Bim and Bmf in BTK inhibitor-sensitive cell lines primed the cells for apoptosis. It was hypothesized that AZD5991, which rapidly inhibits Mcl1 function, would the balance of pro- and anti-apoptotic Bcl2 family proteins toward cell death. Two BTK inhibitor-sensitive cell lines were treated with either AZD5991 alone or following pre-treatment with acalabrutinib. In both BTK inhibitor-sensitive cell lines, the combination resulted in the robust induction of cleaved caspase by 2 hours compared to no positive caspase activation with single agent AZD5991 throughout the 8 hr exposure, as shown in FIG. 2.

### Example 3: Combination of AZD5991 with Acalabrutinib in a BTK Inhibitor-Sensitive In Vivo Model Results in Synergistic Anti-Tumor Activity

5×10⁶ OCILy10 tumor cells were injected subcutaneously in the right flank of C.B.-17 SCID female mice in a volume of 0.1 mL containing 50% matrigel. Tumor volumes (measured by caliper), animal body weight, and tumor condition were recorded twice weekly for the duration of the studies. The tumor volume was calculated using the formula: length (mm)×width (mm)²×0.52. For efficacy studies, growth inhibition from the start of treatment was assessed by comparison of the differences in tumor volume between control and treated groups. Dosing began when mean tumor size reached approximately 150-180 mm³. CR=complete response. AZD5991 was formulated in 30% HPBCD (hydroxypropyl beta-cyclodextrin) in water for injection and adjusted to pH 9.0 up to a concentration of 30 mg/mL. AZD5991 was dosed IV once weekly. Acalabrutinib was formulated in 0.5% hydroxypropyl methyl cellulose/0.2% Tween 80, and dosed twice a day (bid) on an 8/16 hr split as an oral (po) administration at a volume of 10 mL/kg at a dose of 12.5 mg/kg.

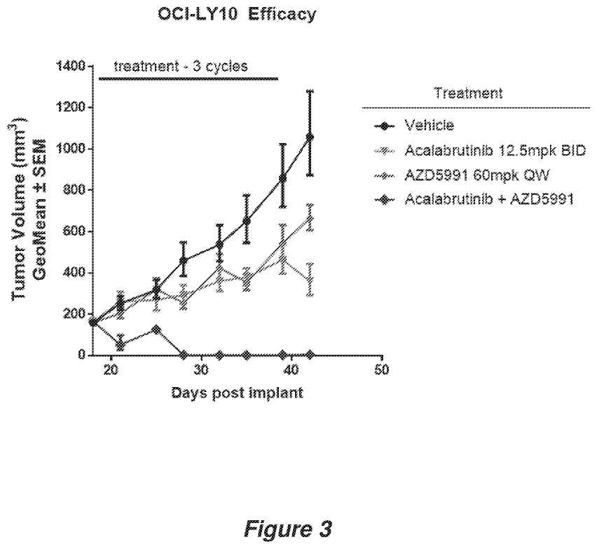

The results of Example 3 are shown in FIG. 3. Combining AZD5991 with acalabrutinib in the ABC-DLBCL model OCI-LY10 produced synergistic anti-tumor activity that resulted in regressions. Twice daily treatment with acalabrutinib yielded 79% tumor growth inhibition, while once weekly administration of AZD5991 as single agent resulted in 44% tumor growth inhibition. Combination of AZD5991 with acalabrutinib resulted in 100% tumor growth inhibition and 98% regressions on day 42. Importantly, all therapies were well tolerated alone and in combination.

1. A method of treating cancer comprising administering to a subject in need thereof an effective amount of 17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (Compound A) or a pharmaceutically acceptable salt thereof; and an effective amount of acalabrutinib, or a pharmaceutically acceptable salt thereof.

2. The method of claim 1, wherein the method comprises administering Compound A or a pharmaceutically acceptable salt separately, sequentially or simultaneously with acalabrutinib.

3. The method of claim 1, wherein acalabrutinib is administered prior to the administration of Compound A, or a pharmaceutically acceptable salt thereof.

4. The method of claim 1, wherein the method comprises administering Compound A.

5. The method of claim 1, wherein the method comprises administering a pharmaceutically acceptable salt of Compound A.

6. The method of claim 1, wherein Compound A is (Rₐ)-17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (AZD5991), or a pharmaceutically acceptable salt thereof.

7. The method of claim 6, wherein the method comprises administering AZD5991.

8. The method of claim 7, wherein the method comprises administering a pharmaceutically acceptable salt of AZD5991.

9. The method of claim 5, wherein the method comprises administering a solid form of AZD5991 or a pharmaceutically acceptable salt thereof.

10. The method of claim 1, wherein the cancer is a hematological cancer.

11. The method of claim 11, wherein the hematological cancer is selected from acute myeloid leukemia (AML), multiple myeloma, mantle cell lymphoma (MCL), chronic lymphocytic leukemia (CLL), diffuse large B cell lymphoma (DLBCL), Burkitt's lymphoma and follicular lymphoma.

12. The method of claim 1, wherein the cancer is a BTK-sensitive cancer.

13-24. (canceled)

25. A kit comprising:
a first pharmaceutical composition comprising acalabrutinib, or a pharmaceutically acceptable salt thereof, and a pharmaceutically acceptable carrier; and
a second pharmaceutical composition comprising Compound A, or a pharmaceutically acceptable salt thereof, and a pharmaceutically acceptable carrier.

26. The kit of claim 25, wherein the second pharmaceutical composition comprises Compound A.

27. The kit of claim 25, wherein the second pharmaceutical composition comprises a pharmaceutically acceptable salt of Compound A.

28. The kit of claim 25, wherein Compound A is (Rₐ)-17-chloro-5,13,14,22-tetramethyl-28-oxa-2,9-dithia-5,6,12,13,22-pentaazaheptacyclo[27.7.1.1^{4,7}.0^{11,15}.0^{16,21}.0^{20,24}.0^{30,35}]octatriaconta-1(37),4(38),6,11,14,16,18,20,23,29,31,33,35-tridecaene-23-carboxylic acid (AZD5991), or a pharmaceutically acceptable salt thereof.

29. The kit of claim 25, wherein the second pharmaceutical composition comprises AZD5991.

30. The kit of claim 28, wherein the second pharmaceutical composition comprises a pharmaceutically acceptable salt of AZD5991.

31. The kit of claim 28, wherein the second pharmaceutical composition comprises a solid form of AZD5991, or a pharmaceutically acceptable salt thereof.
